(19) United States
(12) Patent Application Publication
(10) Pub. No.: US 2020/0165588 A1
(43) Pub. Date: May 28, 2020

(54) METHOD FOR EXTRACTING B-AMYLASES FROM A SOLUBLE FRACTION OF A STARCH PLANT AND IN THE PRESENCE OF A PROTEASE

(71) Applicant: ROQUETTE FRERES, Lestrem (FR)

(72) Inventors: Vincent COURBOIS, Bethune (FR); Pierrick DUFLOT, La Couture (FR); Aline LECOCQ, Bethune (FR); Jean-Marc VERRIN, Beuvry (FR)

(21) Appl. No.: 16/699,832

(22) Filed: Dec. 2, 2019

Related U.S. Application Data
(63) Continuation of application No. 14/419,970, filed on Feb. 6, 2015, now abandoned.

(30) Foreign Application Priority Data
Aug. 7, 2012 (FR) 12 57680

(51) Int. Cl. C12N 9/26

(52) U.S. Cl. CPC C12N 9/2425; C12Y 302/01002; C12N 9/2408

(57) ABSTRACT

The present invention concerns an improved method for extracting β-amylases from a soluble fraction of a starch plant, said method comprising a step of clarification by microfiltration and a step of concentration and purification by ultrafiltration. This method is characterised in that it uses a protease during the microfiltration step, making it possible to greatly reduce the fouling of the membranes used, which increases the production time before washing. Simultaneously, perfectly clear permeates are obtained, which assists with the subsequent ultrafiltration step and makes it possible to improve the transmission of β-amylase. Finally, the protease has no negative effect on the β-amylase activity.

The present invention relates to an improved method for extracting β-amylases from a soluble fraction of a starch plant, said method comprising a step of clarification by microfiltration and a step of concentration/purification by ultrafiltration. This method makes use of a protease during the microfiltration step, which very substantially reduces the fouling of the membranes used: the production time before washing, and therefore the economic efficiency of the corresponding method, are thus increased. In parallel, the use of such a protease results in perfectly clear permeates, which facilitates the ultrafiltration step, and makes it possible to improve the β-amylase transmission. Finally, the protease has no negative effective on the β-amylase activity.

β-amylases are exohydrolases which release maltose units from the nonreducing β-ends of α-(1→4)-linked glucose polymers or oligomers, the reaction stopping at the first point of α-(1→6) branching encountered. Major components of the “diastatic power” (corresponding to the combined activities of α-amylases, β-amylases, α-glucosidases and debranching enzymes) during malting (artificial germination of cereal seeds), the β-amylase activities isolated from this enzymatic cocktail are essential for the production of maltose or of other fermentable sugars generated from starch.

The saccharifying activity of β-amylases alone is therefore exploited in a large number of applications: in breadmaking, in the malt industry, as a food additive, or even as a digestive agent, for the production of sweeteners, in pharmacy for the production of vaccines, and finally for the production of maltose and of maltose-enriched syrups (precursor of maltitol and maltitol syrups).

There are many methods for producing β-amylases. It is thus known that ungerminated barley, rye or wheat seeds are all biological materials of choice for the large-scale commercial preparation of β-amylases. It is, moreover, known to those skilled in the art that half of the β-amylases that can be extracted from ungerminated barley, wheat or rye seeds can be readily obtained in the form of free enzymes by extraction with water and saline solutions. The other half is partly in “bound” form which requires the addition of reducing agents or proteolytic enzymes for the extraction thereof. Another β-amylase fraction that is not directly extractable, termed “latent” fraction, has also been described: detergents are necessary in order to extract it from cereal seeds. Moreover, the β-amylase extraction methods described in the prior art are adapted according to the intended application.

In this regard, the applicant has recently developed and protected in application EP 2 414 379 an original method for producing β-amylases, in the sense that it is based on a starting raw material thus far poorly exploited: the “soluble fractions”. The latter were previously used exclusively as a source of nitrogen in fermentation and as nutritive feed for livestock once said fractions had been enriched in fibers.

These soluble fractions are produced during the wet extraction of the components of starch plants, such as corn, potato, sweet potato, wheat, rice, pea, broad bean, horse bean, cassava, sorghum, konjac, rye, buckwheat and barley. The “noble” components, produced during the extraction, are in particular starches, proteins or else fibers. The “soluble fractions” in contrast denote “non-noble” constituents: they are the liquid residues resulting from said extraction, even though such residues may still contain trace amounts of the insoluble substances and also various and varied particles and colloids.

The method which is the subject of application EP 2 414 379 is based on the initial selection of the soluble fraction to be treated, and then on a step of clarification of said fraction carried out by microfiltration. On this occasion, it was demonstrated that the β-amylase obtained was particularly suitable for the preparation of maltose syrups, in the same way as a β-amylase produced according to prior techniques but using more complex and more expensive methods.

While continuing its development studies on this technology, the applicant has been confronted with a new technical problem: it in fact proves to be the case that the residual insoluble substances, the particles and the colloids contained in the “soluble fractions” used, behave like “poisons” against the microfiltration membranes by blocking said membranes. The productivity of the method is then reduced by the interruptions made obligatory by rapid pressure increase: cleaning therefore proves to be obligatory. In other words, the method which is the subject of application EP 2 414 379 needs to be improved so as to solve the problem of fouling of the microfiltration membranes.

Working to that effect, the applicant has succeeded in demonstrating that the use of a protease, during the step of clarification by microfiltration, very substantially reduces the fouling problems. Entirely surprisingly, it proves to be the case that said protease does not in any way affect the properties of the β-amylase: as is well known, when a protease is used in a medium containing proteins, including a protein of interest, said protease is capable of degrading the protein of interest.

Extremely clear permeates are obtained according to the invention, and the transmission of the β-amylase activity is improved. Those skilled in the art have therefore been successfully provided with a method for producing β-amylases which are perfectly suitable for the synthesis of maltose syrups, through a method that is simple to carry out, while at the same time greatly limiting the problem of fouling of microfiltration devices.

Thus, a first subject of the present invention consists of a method for extracting β-amylases from a soluble fraction of a starch plant, comprising:
a) a step of microfiltration of a soluble fraction of a starch plant,
b) followed by an ultrafiltration step, said method being characterized in that the microfiltration step is carried out in the presence of at least one protease.

The proteases (or peptidases or proteolytic enzymes) are enzymes which break the peptide bonds of proteins. The term “proteolytic cleavage” or “proteolysis” is then used. This process involves the use of a water molecule, which classes them among the hydrolases. Proteases have varied biological functions: they are involved in protein maturation, in food digestion, in blood coagulation, in tissue remodeling during the development of the organism and in healing.

There are four major families of proteases according to the nature of the amino acid(s) of the active site involved in the catalysis:
serine proteases: digestive enzymes in mammals (for example: alcalase),
thiol proteases which have a cysteine in their active site: papain (Lypaine™ 6500 L), bromolain, cathepsins,
aspartyl proteases: acid proteases which act at acidic pH and have an aspartic acid on their active site (for example: pepsin),
metalloproteases which have a metal cation (Zn²⁺) which intervenes to activate a water molecule which cleaves the peptide chain (Neutrase, Brewlyve™ NP 900).

The soluble fraction of starch plants to be treated should be chosen upstream of the method in accordance with the invention. This selection is made in particular from the group consisting of the soluble fractions of wheat, of potato, of pea, of broad bean, of horse bean, of rice, of barley, of rye, of buckwheat and of sweet potato, and preferentially of wheat and of barley.

The first step of the method in accordance with the invention consists in carrying out a step of microfiltration of said soluble fractions in the presence of at least one protease. The objective of the microfiltration is in particular to remove the insoluble substances, the colloids and the microbiological material for the purpose of obtaining a clear composition containing β-amylase. The latter composition is therefore the microfiltration permeate. Prior to the microfiltration, the protease is brought into contact with the soluble fraction of a starch plant to be treated: those skilled in the art will know how to adjust the contact time required for the action of the enzyme.

The protease used in the present invention is chosen from serine proteases, thiol proteases, aspartyl proteases and metalloproteases, and is more particularly chosen from metalloproteases. By name, the proteases that are preferred in the present invention are the products sold under the names: Sumizymen™ APL, Lypaine™ 6500 L, Neutrase™ 0.8 L, Brewlyve™ NP 900 and Brewers Clarex™. Use will preferably be made of an amount of protease of between 0.01% and 0.1% by volume relative to the volume of the soluble fraction of a starch plant to be treated.

The microfiltration step of the method in accordance with the invention is preferentially carried out by tangential membrane microfiltration. More particularly, the applicant company recommends carrying out the tangential microfiltration with ceramic membranes having a porosity of 0.1 μm to 1 μm.

Optionally, the microfiltration step can be preceded by a step of flocculation of the insoluble particles contained in the soluble fraction of starch plants, by any technique known moreover to those skilled in the art.

For this first microfiltration step, the applicant recommends working at a pH of between 4 and 5 and at a temperature of between 40° C. and 50° C.

The microfiltration step is in particular controlled by the increase in the transmembrane pressure (TMP) over time, at a fixed permeate flow rate. The turbidity of the permeate is caused by colloidal particles which absorb, scatter and/or reflect light, but especially by the protein-polyphenol complexes. It can be determined by means of a turbidity meter, but can also be very well assessed visually. With regard to the TMP pressure, it is determined conventionally by means of sensors placed at the inlet and at the outlet of the filtration device.

The enzymatic activity of the solution containing the β-amylase is also evaluated. The enzymatic activity measurement is determined through the diastatic activity. The latter is expressed in degrees of diastatic power (° DP), defined as the amount of enzyme contained in 0.1 ml of a 5% solution of a sample of enzyme preparation sufficient to reduce 5 ml of Fehling's solution, when said sample is placed in 100 ml of the substrate for 1 h at 20° C. Since the diastatic power makes it possible to measure all the enzymatic activities of amylase type, the Applicant company made sure by means of other, more specific enzymatic methods (for example using the MEGAZYME assay kit specific for α-amylase, sold by CERALPHA METHOD) that the preparation of β-amylases in accordance with the invention did not contain other contaminating activities.

The transmission is then determined as being the ratio of β-amylase activity contained in the microfiltration permeate to the β-amylase activity contained in the microfiltration feed.

The second step of the method according to the invention is an ultrafiltration step, aiming first of all to concentrate the microfiltration permeate containing the β-amylase, while at the same time removing from it any contaminating residual salts, sugars and proteins. The ultrafiltration is thus carried out on the microfiltration permeate so as to obtain an ultrafiltration retentate containing the β-amylase. The ultrafiltration retentate is then dialyzed so as to reduce the concentration of impurities in said retentate.

More particularly, the applicant company recommends carrying out the ultrafiltration using membranes which have a cut-off threshold of 10 000 Da to 50 000 Da, preferably a threshold of 30 000 Da. The soluble fractions are ultrafiltered on a module equipped with polysulfone membranes having a cut-off threshold of 30 000 Da in cassettes on a laboratory scale and polysulfone spiral membranes having a cut-off threshold of 30 000 Da on a pilot scale. The enzyme then becomes concentrated in the retentate over the course of time.

Finally, a second subject of the present invention consists of the use of proteases in a method for extracting β-amylases from a soluble fraction of a starch plant.

The examples which follow make it possible to understand the invention more clearly, without however limiting the scope thereof.

### EXAMPLES

In this example, the preparation of β-amylase is first of all carried out according to the method described in application EP 2 414 379, i.e. on the basis of a step of microfiltration of soluble fraction without using protease: it is the control test. The preparation of β-amylase is then carried out according to the method which is the subject of the present invention, i.e. by microfiltration of soluble fraction in the presence of a protease: these tests illustrate the present invention.

In the manufacture of starch from wheat, a soluble fraction is collected at the inlet of the solubles evaporator, a step conventionally carried out to produce products intended for feeding livestock, once concentrated. These products are sold by the applicant company under the name CORAMI®. These soluble fractions have a pH of 4 and a β-amylase activity of about 30° DP.

The microfiltration of soluble fractions of wheat is carried out here on pilot-scale equipment. The microfiltration unit is equipped with ceramic membranes made of titanium oxide, the cut-off threshold of which is equal to 0.2 μm. The permeate flow rate is fixed at 12 l/h m². The volume concentration factor is equal to 1.5. The temperature and the pH of the permeate are respectively equal to 45° C. and 4.5.

The test protease is added to the soluble fraction, said test protease being at a concentration fixed at 0.1% by volume relative to the total volume of said composition. This protease is left to act beforehand for 1 hour.

For each of the tests, the change in the TMP pressure is monitored, as already previously described, during the microfiltration step: the TMP increases over time as the membrane becomes fouled. For each test, the degree of diastatic power (° DP) is also determined at the beginning of the test (Feed ° DP), after 1 hour (° DP 1 hour) and at the end of the method after approximately 8 hours (° DP 8 hours); it is thus possible to calculate the transmission of the β-amylase activity after 1 hour (T 1 hour) and after 8 hours (T 8 hours). Finally, the turbid or clear nature of the permeate is determined at the end of each test, visually.

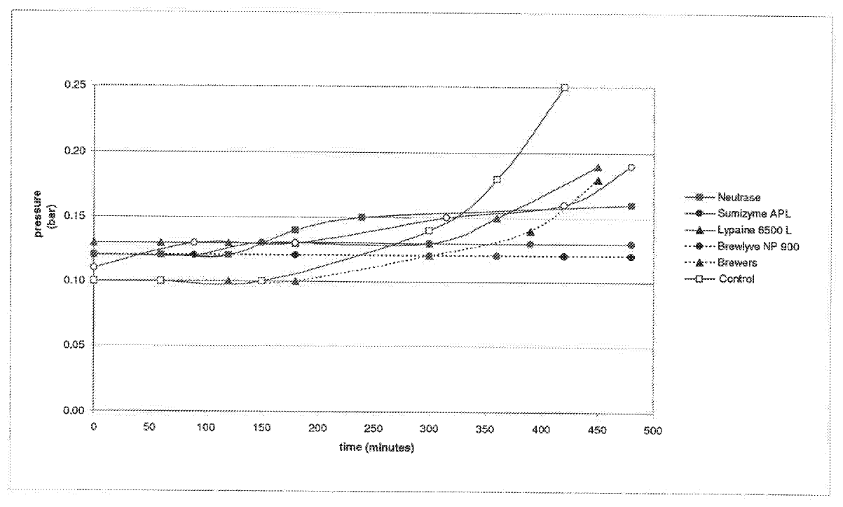

FIG. 1/1 illustrates the change in the TMP pressure as a function of time, for the control test carried out without protease, and for each test representative of the invention which uses a protease. It is very clearly apparent that the use of a protease makes it possible to limit the increase in the TMP pressure over time: the membrane fouling phenomena have therefore indeed been successfully slowed down. It is even noted that, for some proteases, including in particular the Brewlyne™ NP 900 product, the transmembrane pressure is perfectly stable: in this case, the membrane fouling phenomenon is virtually absent.

Furthermore, it is noted for each test according to the invention that the permeate obtained is perfectly clear. Finally, as demonstrated in table 1, the transmission is improved in the case of the invention compared with the control test carried out without protease.

TABLE 1

| Protease | Feed | ° DP 1 | T 1 | ° DP 8 | T 8 |
|---|---|---|---|---|---|
| used | ° DP | hour | hour | hours | hours |
| None | 31 | 25 | 81 | 19 | 61 |
| (control) | | | | | |
| Sumizyne ™ | 31 | 28 | 90 | 22 | 71 |
| APL | | | | | |
| Lypaine ™ | 38 | 33 | 87 | 26 | 68 |
| 6500 L | | | | | |
| Brewlyne ™ | 36 | 32 | 89 | 27 | 75 |
| NP 900 | | | | | |
| Brewers | 37 | 35 | 95 | 25 | 68 |
| Clarex ™ | | | | | |
| Neutrase ™ | 32 | 27 | 84 | 24 | 75 |
| 0.8 L | | | | | |

The microfiltration step is followed by an ultrafiltration step, carried out on the microfiltration permeate. The main objective thereof is to concentrate said permeate and to remove from it any contaminating residual salts, sugars and proteins. 40 liters of microfiltration permeate are recovered for each of the tests apart from the control test. Said permeate is ultrafiltered on a MILLIPORE laboratory module with 0.18 m² of membranes having a cut-off threshold of 30 000 Da. 39.5 liters of ultrafiltered permeate and a retentate concentrated by a factor of 75, having a β-amylase activity of between 1500 and 1600° DP, are recovered. The ultrafiltration retentate is continuously dialyzed at constant volume with 2.5 volumes of water so as to reduce the concentration of impurities of the solubles by a factor of 10. The β-amylase preparation thus obtained is then stored at +4° C.

### Example 2

The same procedure as previously is carried out, but starting from soluble fractions having a pH of 4 and a β-amylase activity of about 30° DP.

The test enzyme is added to the soluble fraction:
Neutrase 0.8 L (protease) at a concentration fixed at 0.1% by volume relative to the total volume of said composition;
Optiflow (hemicellulase) at a concentration fixed at 1% by volume relative to the total volume of said composition;
Rapidase (pectinase) at a concentration fixed at 1% by volume relative to the total volume of said composition;
Finizyme (lysophospholipase) at a concentration fixed at 1% by volume relative to the total volume of said composition.

Each enzyme is left to act beforehand for 1 h.

For each test, the degree of diastatic power (° DP) is also determined at the beginning of the test (Feed ° DP), after 1 hour (° DP 1 hour) and at the end of the method after approximately 8 hours (° DP 8 hours); it is thus possible to calculate the transmission of the β-amylase activity after 1 hour (T 1 hour) and after 8 hours (T 8 hours). The results appear in table 2.

It is noted that the protease according to the invention makes it possible to improve the transmission of the β-amylase activity much more significantly than the other enzymes. In addition, after 8 hours, only the permeate obtained with the protease according to the invention is clear, the others having a very marked cloudy appearance.

TABLE 2

| Enzyme | Feed | ° DP 1 | T 1 | ° DP 8 | T 8 |
|---|---|---|---|---|---|
| used | ° DP | hour | hour | hours | hours |
| None | 32 | 25 | 79 | 21 | 70 |
| (control) | | | | | |
| Neutrase ™ | 32 | 29 | 92 | 25 | 86 |
| 0.8 L | | | | | |
| Optiflow ™ | 31 | 20 | 65 | 9 | 30 |
| Rapidase ™ | 31 | 21 | 68 | 12 | 39 |
| Finizyme ™ | 31 | 26 | 84 | 23 | 74 |

1-10. (canceled)

11. A method for extracting β-amylases from a soluble fraction of a wheat, comprising the steps of:
a) microfiltration of a soluble fraction of a wheat; and
b) an ultrafiltration step, wherein the microfiltration step is carried out in the presence of at least one protease.

12. The method according to claim 11, wherein the protease is chosen from serine proteases, thiol proteases, aspartyl proteases and metalloproteases.

13. The method according to claim 12, wherein the protease is a metalloprotease.

14. The method according to claim 11, wherein microfiltration step is carried out by tangential membrane microfiltration.

15. The method according to claim 14, wherein the tangential microfiltration is carried out with ceramic membranes having a porosity of 0.1 μm to 1 μm.

16. The method according to claim 11, wherein the microfiltration step is preceded by a step of flocculation of the insoluble particles contained in the soluble fraction of starch plants.

17. The method according to claim 11, wherein the microfiltration step is carried out at a pH of between 4 and 5 and at a temperature of between 40° C. and 50° C.

18. The method according to claim 11, wherein the ultrafiltration is carried out using membranes which have a cut-off threshold of 10 000 Da to 50 000 Da.

19. The method according to claim 18, wherein the ultrafiltration is carried out using polysulfone membranes having a cut-off threshold of 30 000 Da in cassettes on a laboratory scale and polysulfone spiral membranes having a cut-off threshold of 30 000 Da on a pilot scale.

20. The method according to claim 18, wherein the ultrafiltration step is carried out using membranes which have a cut-off threshold of 30 000 Da.
